(19) United States
(12) Patent Application Publication
(10) Pub. No.: US 2023/0381524 A1
(43) Pub. Date: Nov. 30, 2023

(54) SYSTEMS AND METHODS FOR CARDIAC CONDUCTION SYSTEM

(71) Applicant: Singular Medical (USA) Inc., Irvine, CA (US)

(72) Inventors: Quan Ni, Irvine, CA (US); Martin Tze, Irvine, CA (US); Scott Hayden, Irvine, CA (US); Entao Liu, Irvine, CA (US); Yongxing Zhang, Irvine, CA (US); Lichuan Ping, Irvine, CA (US); Weiguang Shao, Irvine, CA (US); Hao Ding, Irvine, CA (US)

(21) Appl. No.: 17/804,767

(22) Filed: May 31, 2022

(51) Int. Cl. A61N 1/37; A61N 1/05; A61N 1/368; A61N 1/375; A61N 1/365; A61B 5/00

(52) U.S. Cl. CPC A61N 1/3706; A61N 1/0597; A61N 1/36843; A61N 1/37512; A61N 1/36507; A61B 5/686

(57) ABSTRACT

Systems and methods for determining parameters and/or configurations for implantable pulse generator and/or lead(s) for the cardiac conduction system. The method includes configuring a stimulation vector. The method also includes configuring an AV delay to be less than an intrinsic AV delay, controlling a pulse generator to deliver pacing with a pacing amplitude, determining a sensing signal from artifacts of the pacing, adjusting the pacing amplitude based on the determined sensing signal and a capture signal, and determining the pacing threshold based on the adjusted pacing amplitude. The method further includes configuring the AV delay to a percentage of an intrinsic AV delay, controlling the pulse generator to deliver pacing with the AV delay, determining a sensing signal from artifacts of the pacing, adjusting the AV delay based on the determined sensing signal, and configuring the pulse generator with the adjusted AV delay.

### FIELD

This disclosure relates generally to systems and methods for a cardiac conduction system. More specifically, the disclosure relates to systems and methods of determining parameters and/or configurations such as sensing and/or pacing configurations (e.g., AV delay, pacing threshold, or the like) for an implantable pulse generator and/or lead(s) for the cardiac conduction system.

### BACKGROUND

An implantable pulse generator (e.g., an implantable pacemaker, an implantable cardioverter-defibrillator, etc.) is a medical device powered by a battery, contains electronic circuitry having a controller, and delivers and regulates electrical impulses to an organ or a system such as the heart, the nervous system, or the like. A lead is a thin, flexible, electrical wire connecting a device such as the implantable pulse generator to a target such as the organ or system, transmits electrical impulses (e.g., a burst of energy) from the device to the target, and/or senses or measures the potential or the voltage from the target. The conduction system of the heart consists of cardiac muscle cells and conducting fibers that are specialized for initiating impulses and conducting the impulses through the heart. The cardiac conduction system initiates the normal cardiac cycle, coordinates the contractions of cardiac chambers, and provides the heart its automatic rhythmic beat. Conduction system pacing (CSP) is a technique of pacing that involves implantation of pacing leads along different sites or pathways of the cardiac conduction system and includes His-bundle pacing, left bundle branch pacing, right bundle branch pacing, and/or bilateral pacing (pacing both the left bundle branch and the right bundle branch).

### SUMMARY

This disclosure relates generally to systems and methods for a cardiac conduction system. More specifically, the disclosure relates to systems and methods of determining parameters and/or configurations such as sensing and/or pacing configurations (e.g., AV delay, pacing threshold, or the like) for an implantable pulse generator and/or lead(s) for the cardiac conduction system.

In an embodiment, an implantable pulse generator for a cardiac conduction system is provided. The pulse generator includes an enclosure containing an electronic circuitry having a controller. The controller is configured to configure a stimulation vector for a lead, configure an AV delay to a first delay that is less than an intrinsic AV delay, control the pulse generator to deliver pacing with a pacing amplitude, determine a sensing signal from artifacts of the pacing, adjust the pacing amplitude by a first amplitude based on the determined sensing signal and a capture signal, and determine a pacing threshold based on the adjusted pacing amplitude. The lead includes a first electrode and a second electrode. The first electrode is positioned at or near left bundle branch of the cardiac conduction system. The second electrode is positioned at or near right bundle branch of the cardiac conduction system.

In an embodiment, a method of determining a pacing threshold for cardiac conduction system is provided. The method includes configuring a stimulation vector for a lead, configuring an AV delay to a first delay that is less than an intrinsic AV delay, controlling a pulse generator to deliver pacing with a pacing amplitude, determining a sensing signal from artifacts of the pacing, adjusting the pacing amplitude by a first amplitude based on the determined sensing signal and a capture signal, and determining the pacing threshold based on the adjusted pacing amplitude. The lead includes a first electrode and a second electrode. The first electrode is positioned at or near left bundle branch of the cardiac conduction system. The second electrode is positioned at or near right bundle branch of the cardiac conduction system.

In an embodiment, an implantable pulse generator for cardiac conduction system is provided. The pulse generator includes an enclosure containing an electronic circuitry having a controller. The controller is configured to configure a stimulation vector for a lead, configure an AV delay to a fraction or percentage of an intrinsic AV delay, control the pulse generator to deliver pacing with the AV delay, determine a sensing signal from artifacts of the pacing, adjust the AV delay by a first delay based on the determined sensing signal, and configure the pulse generator with the adjusted AV delay. The lead includes a first electrode and a second electrode. The first electrode is positioned at or near left bundle branch of the cardiac conduction system. The second electrode is positioned at or near right bundle branch of the cardiac conduction system.

In an embodiment, a method of determining a pacing threshold for cardiac conduction system is provided. The method includes configuring a stimulation vector for a lead, configuring the AV delay to a fraction or percentage of an intrinsic AV delay, controlling the pulse generator to deliver pacing with the AV delay, determining a sensing signal from artifacts of the pacing, adjusting the AV delay by a first delay based on the determined sensing signal, and configuring the pulse generator with the adjusted AV delay. The lead includes a first electrode and a second electrode. The first electrode is positioned at or near left bundle branch of the cardiac conduction system. The second electrode is positioned at or near right bundle branch of the cardiac conduction system.

Embodiments disclosed herein can help to determine optimal parameters and configurations for the pulse generator and/or lead(s) to e.g., minimize the electrical energy used and/or to shorten the total ventricular activation time. Embodiments disclosed herein can also help to predict and estimate parameters and configurations for the pulse generator and/or lead(s) for optimal personalized therapy using e.g., machine learning.

Other features and aspects will become apparent by consideration of the following detailed description and accompanying drawings.

### BRIEF DESCRIPTION OF THE DRAWINGS

References are made to the accompanying drawings that form a part of this disclosure and which illustrate the embodiments in which systems and methods described in this specification can be practiced.

FIG. 1 is a perspective view of a lead being inserted in the ventricular septum, according to an embodiment.

FIG. 2 is a perspective view of a lead being inserted in the ventricular septum, according to another embodiment.

FIG. 3 is a flow chart illustrating a method of determining a pacing threshold for the cardiac conduction system, according to an embodiment.

FIG. 4 is a flow chart illustrating a method of determining an AV delay for the cardiac conduction system, according to an embodiment.

FIG. 5 illustrates a schematic view of a method for a machine learning system for predicting and/or estimating parameters and/or configurations for the pulse generator and/or lead(s) for the cardiac conduction system, according to an embodiment.

Particular embodiments of the present disclosure are described herein with reference to the accompanying drawings; however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. In this description, as well as in the drawings, like-referenced numbers represent like elements that may perform the same, similar, or equivalent functions.

### DETAILED DESCRIPTION

This disclosure relates generally to systems and methods for a cardiac conduction system. More specifically, the disclosure relates to systems and methods of determining parameters and/or configurations such as sensing and/or pacing configurations (e.g., AV delay, pacing threshold, or the like) for an implantable pulse generator and/or lead(s) for the cardiac conduction system.

As defined herein, the phrase “pacing” or “stimulation” may refer to depolarization of the atria or ventricles, resulting from an impulse delivered (e.g., at desired voltage(s) for a desired duration, or the like) from a device (such as a pulse generator) down a lead to the heart. Cardiac pacing typically involves the delivery of a polarizing electrical impulse from an electrode of a lead in contact with the myocardium, together with the generation of an electrical field of sufficient intensity to induce a propagating wave of cardiac action potentials. It will be appreciated that cardiac myocytes can be “activated” by the delivery of an electrical pacing stimulus. The pacing stimulus can create an electrical field that allows for the generation of a self-propagating wavefront of action potentials that may then advance from the stimulation site. For the pacing stimulus to produce a wave of depolarization in a cardiac chamber (which may be referred to as “capture”), the pacing stimulus must exceed a critical amplitude (measured in volts or milliamperes) and must be applied for a sufficient duration. If the pacing stimulus is not of sufficient amplitude or duration, it may not initiate such a wavefront. The minimum amplitude (i.e., stimulus intensity) and duration required to reliably initiate a propagated depolarizing wavefront or to generate the self-propagating wavefront that results in cardiac activation may be referred to as the “threshold” or “stimulation threshold”. As defined herein, the phrase “pacing threshold” may be referred to as a pacing output that is programmed to exceed the stimulation threshold by an adequate margin (referred to as a “safety margin”) to reduce the risk of loss of capture during fluctuations.

As defined herein, the phrase “sensing” may refer to detection by the device of intrinsic or stimulated atrial or ventricular depolarization signals that are conducted up a lead. It will be appreciated that myocardial capture can be confirmed by detecting stimulated myocardial depolarization (which may be referred to as “evoked response”, which is the electrical event that results from myocardial capture after a pacing output pulse) following pacing. Sensing also includes the ability of the pulse generator to detect the presence or absence of an evoked response. The evoked response can also be referred to as the electrical signature, or artifacts, of the pacing signal which may include small, narrow electrical pulses.

As defined herein, the phrase “morphology” or “wave form morphology” may refer to the shape, amplitude, and/or duration of the potentials which are the electrical signals in an electrocardiogram (or electrogram). As defined herein, the phrase “QRS” or “QRS complex” includes the Q wave, the R wave, and the S wave and may refer to the electrical impulse as it spreads through the ventricles and indicates ventricular depolarization. It will be appreciated that whenever electrocardiogram is referred to in this specification, electrogram may apply as well.

It will be appreciated that after an electrical impulse is generated by the sinus node of the heart, the electrical impulse can spread across both atria, causing these chambers to beat. The atrioventricular (AV) node then “gathers” that electrical impulse and, after a brief delay (may referred to as “AV delay”), allows the electrical impulse to pass through to the ventricles. Such AV delay is referred to as an “intrinsic AV delay”. AV delay in the transmission of the electrical signal through the AV node is critical to a normal heartbeat and the efficient functioning of the heart. When a pacing (e.g., a ventricular pacing) is needed, AV delay can be configured or programmed to determine when a ventricular pacing needs to be conducted for the efficient functioning of the heart. Such AV delay is referred to as an “AV delay” or “programmable AV delay”.

As defined herein, the phrases “near-field” and “far-field” may refer to regions of potentials due to depolarization of the heart tissue. Near-field (or “local”) may refer to the region close to an object (e.g., the sensing electrode, or the like), while far-field may refer to the region at greater distances.

It will be appreciated that two electrodes, a cathode and an anode, are required to complete the electrical circuit between the body and the pulse generator. In a bipolar system, both anode and cathode are located in the heart, whereas in a unipolar system only the cathode is located in the heart and the pulse generator can serve as the anode or ground. It will also be appreciated that the body (typically made of metal) of the pulse generator may be referred to as a “can”. As defined herein, the phrase “vector” of sensing or pacing (i.e., sensing vector or pacing vector) may refer to a direction of sensing or pacing, depending upon the relative proximity of the electrodes being used in the pacing and/or sensing. It will further be appreciated that a stimulation vector can be referred to as a pacing vector and/or a sensing vector.

It will be further appreciated that differences in inter-electrode distance between bipolar and unipolar leads can have an important influence on sensing of “far-field” or “near-field” signals. For example, due to the much smaller field of view, bipolar electrodes tend to be minimally influenced by electrical signals that originate outside the heart, while unipolar leads may detect electrical signals that originate from “near-field” and/or “far-field”.

As defined herein, the phrase “distal” may refer to being situated away from a point of attachment (e.g., to a device such as the implantable pulse generator) or from an operator (e.g., a physician, a user, etc.). A distal end of a lead or a catheter may refer to an end of the lead or the catheter that is away from the operator or from a point of attachment to the implantable pulse generator.

As defined herein, the phrase “proximal” may refer to being situated nearer to a point of attachment (e.g., to a device such as the implantable pulse generator) or to an operator (e.g., a physician, a user, etc.). A proximal end of a lead or a catheter may refer to an end of the lead or the catheter that is close to the operator or to a point of attachment to the implantable pulse generator.

As defined herein, the phrase “French” may refer to a unit to measure the size (e.g., diameter or the like) of device such as a catheter, a lead, etc. For example, a round catheter or lead of one (1) French has an external diameter of ⅓ millimeters. For example, if the French size is 9, the diameter is 9/3=3.0 millimeters.

As defined herein, the phrase “helix” may refer to (e.g., an object) having a three-dimensional shape like that of a wire wound (e.g., in a single layer) around a cylinder or cone, as in a corkscrew or spiral staircase. The phrase “linear” may refer to being arranged in or extending straightly or nearly straightly.

As defined herein, the phrase “conductive” may refer to electrically conductive.

As defined herein, the phrase “septum” may refer to a partition separating two chambers, such as that between the chambers of the heart. Septum can be atrial septum and/or ventricular septum. The phrase “ventricular septum” or “inter-ventricular septum” may refer to a partition separating two ventricular chambers. The phrase “right ventricular septum” may refer to the ventricular septum where the RBB is located, while “left ventricular septum” may refer to the ventricular septum where the LBB is located.

As defined herein, the phrase “conduction system pacing” or “CSP” may refer to a therapy that involves the placement of permanent pacing leads along different sites or pathways of the cardiac conduction system (i.e., the heart's electrical conduction system) with the intent of overcoming sites of atrioventricular conduction disease and delay, providing a pacing solution that results in more synchronized biventricular activation. Lead placement for CSP can be targeted at the bundle of His, known as His-bundle pacing (HBP), at the region of the left bundle branch (LBB), known as LBB pacing (LBBP), or at the region of the right bundle branch (RBB), known as RBB pacing (RBBP). Compared with conventional right ventricular (RV) pacing or biventricular (RV and left ventricular (LV)) pacing, where RV apical pacing lead and/or LV epicardial lead are implanted, the lead for CSP is placed through the septum e.g., closer to the main trunk of the His-bundle, the LBB, and/or the RBB. As such, the design, function, and purpose of the lead(s) for cardiac conduction system are different from those of the lead(s) for RV and/or LV pacing. It will be appreciated that ventricular pacing (e.g., RV pacing or the like) may be un-physiological and may result in adverse outcomes of mitral and/or tricuspid regurgitations, atrial fibrillation, heart failure, and/or pacing induced cardiomyopathy. CSP can be physiological pacing that can results in electrical-mechanical synchronization to mitigate chronic clinical detrimental consequence including e.g., pacing induced cardiomyopathy.

It will be appreciated that when a patient has left bundle branch block (LBBB), the LBB of the cardiac conduction system may be partially or completely blocked, which may cause the left ventricle to contract a little later than it should. In such case, LBBP may be needed. When a patient has right bundle branch block (RBBB), which is an obstacle in the RBB of the cardiac conduction system that makes the heartbeat signal late and out of sync with the LBB, creating an irregular heartbeat. In such case, RBBP may be needed.

It will also be appreciated that CSP indications may include e.g., a high burden of ventricular pacing being necessary (e.g., permanent atrial fibrillation with atrioventricular block, slowly conducted atrial fibrillation, pacing induced cardiomyopathy, atrioventricular node ablation, etc.); sick sinus syndrome, when atrioventricular node conduction diseases exist; and/or an alternative to biventricular pacing in heart failure patients with bundle branch block, narrow QRS and PR prolongation, biventricular pacing no-responders or patients need biventricular pacing cardiac resynchronization therapy upgrade, or the like.

Some embodiments of the present application are described in detail with reference to the accompanying drawings so that the advantages and features of the present application can be more readily understood by those skilled in the art. The terms “near”, “far”, “top”, “bottom”, “left”, “right”, and the like described in the present application are defined according to the typical observation angle of a person skilled in the art and for the convenience of the description. These terms are not limited to specific directions.

Processes described herein may include one or more operations, actions, or functions depicted by one or more blocks. It will also be appreciated that although illustrated as discrete blocks, the operations, actions, or functions described as being in various blocks may be divided into additional blocks, combined into fewer blocks, or eliminated, depending on the desired implementation. Any features described in one embodiment may be combined with or incorporated/used into the other embodiment, and vice versa. The scope of the disclosure should be determined by the appended claims and their legal equivalents, rather than by the examples given herein. For example, the steps recited in any method claims may be executed in any order and are not limited to the order presented in the claims. Moreover, no element is essential to the practice of the disclosure unless specifically described herein as “critical” or “essential.”

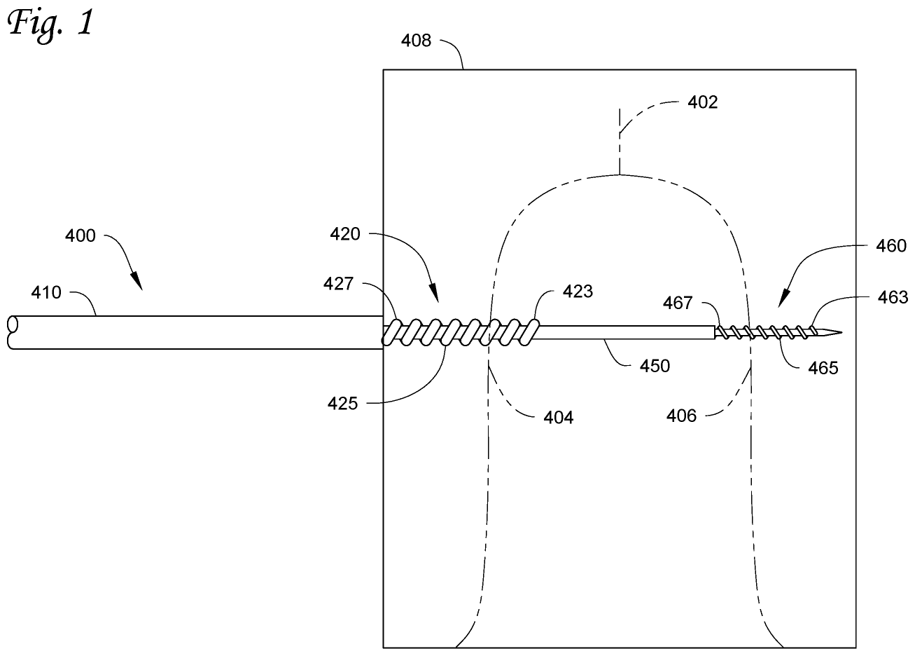

FIG. 1 is a perspective view of a lead 400 being inserted in the ventricular septum 408, according to an embodiment. The cardiac conduction system includes conduction pathways including the His-bundle 402, the RBB 404, and/or the LBB 406.

As shown in FIG. 1, the lead 400 includes a lead body 410 and a distal end including a first electrode 460 having a tapered tip and a rod integral to the tapered tip, a spacer 450 connected to the first electrode 460, and a second electrode 420 fixed to the lead body 410. In an embodiment, the first electrode 460 can include a proximal coated (with non-conductive material, for electrical insulation) region and an uncoated tip (which serve as electrode(s)). The proximal end of the lead is not shown.

In an embodiment, the first electrode 460 can be a linear electrode. In another embodiment, the first electrode 460 can be an auger electrode such as a helix auger where an outer surface of a rod and/or a tip of a linear electrode is threaded for deep seating the first electrode 460. In an embodiment, the second electrode 420 is a helix electrode. In an embodiment, the spacer 450 can be fixed (e.g., a distance between the first electrode 460 and the second electrode 420 can be fixed). In another embodiment, the spacer 450 can be adjustable to distally extend or proximally retract the first electrode 460, so that the spacing between the first electrode 460 and the second electrode 420 can vary (to provide variable spacing between the first electrode 460 and the second electrode 420). A length of the spacer 450 (i.e., a distance between the first electrode 460 and the second electrode 420 when the lead is fully deployed) can be at or about four millimeters.

In an embodiment, the first electrode 460 can be a single polar electrode (e.g., cathode). In an embodiment, the first electrode 460 can be a bipolar electrode including e.g., a distal cathode 463, a non-conductive middle portion 465, and a proximal anode 467. The first electrode 460 can have a length of at or about four millimeters. The distal cathode 463 of the first electrode 460 can have a length of at or about one millimeter, the non-conductive middle portion 465 can have a length of at or about two millimeters, and the proximal anode 467 can have a length of at or about one millimeter.

In an embodiment, the second electrode 420 can be a single polar electrode (e.g., anode). In an embodiment, the second electrode 420 can be a bipolar electrode including e.g., a distal cathode 423, a non-conductive middle portion 425, and a proximal anode 427. The second electrode 420 can have a length of at or about four millimeters. The distal cathode 423 of the second electrode 420 can have a length of at or about one millimeter, the non-conductive middle portion 425 can have a length of at or about two millimeters, and the proximal anode 427 can have a length of at or about one millimeter.

In another embodiment, the electrode 420 can be a bipolar electrode including e.g., a distal portion and a proximal portion. The distal portion of the electrode 420 can be non-conductive (e.g., used as fixation) and the proximal portion can be conductive (e.g., used as electrode). In such embodiment, the electrode 420 can have a length of at or about four millimeters, the distal portion of the electrode 420 can have a length of at or about two millimeters, and the proximal portion can have a length of at or about two millimeters.

In an embodiment, an outer diameter of the first electrode 460 ranges from at or about three French to at or about five French. In an embodiment, the lead 400 can include a ring electrode.

As shown in FIG. 1, the second electrode 420 is disposed/placed at or near the RBB 404, the first electrode 460 is disposed/placed at or near the LBB 406. It will be appreciated that optimal electrical performance can be achieved when the electrode(s) are disposed/placed at the desired location (e.g., at or near the conduction pathway(s) that electrical impulses (e.g., a burst of energy) are to be delivered or sensing signals are to be detected). It will also be appreciated that a thickness of the ventricular septum 408 (e.g., in a direction from the left to the right) can be at or about 13 millimeters. It will further be appreciated that the thickness of the ventricular septum 408 can vary from person to person. For example, the older the person is, typically the thicker the ventricular septum 408 is. It will also be appreciated that the closer the electrode of the lead is to the RBB/LBB, the lower the voltage it may take to provide cardiac conduction system stimulations, and thus the lead needs to go deeper to the ventricular septum 408.

In an embodiment, the spacer 450 extends from a distal tip of the lead body 410. An outer diameter of the spacer 450 can be smaller than an inner diameter of the second electrode 420 (the helix electrode), such that the spacer and the second electrode 420 are co-axial, and/or that the spacer 450 is disposed in the helical space of the second electrode 420. In an embodiment, the second electrode 420 can wrap around (and in contact with) the spacer 450.

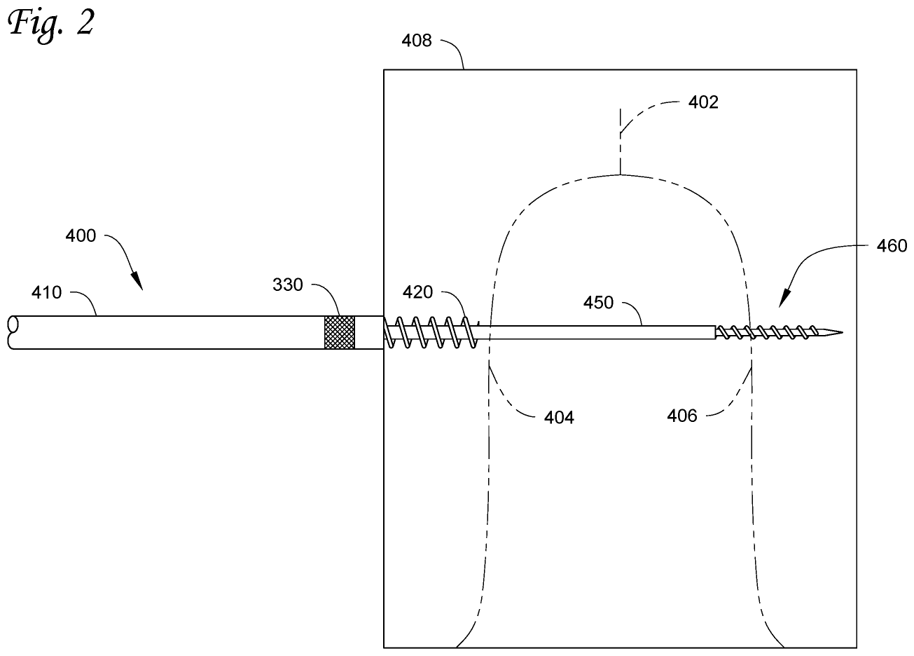

FIG. 2 is a perspective view of a lead 400 being inserted in the ventricular septum 408, according to another embodiment. It will be appreciated that the lead of FIG. 2 can be the same as or similar to the lead of FIG. 1, unless explicitly specified otherwise. As shown in FIG. 2, the lead 400 includes a ring electrode 330 disposed around the lead body 410 proximal to the helix electrode 420. The ring electrode 330 can be disposed outside (e.g., in contact with, or separate from) the ventricular septum 408 (e.g., in the right ventricle). In another embodiment, the ring electrode 330 can be disposed inside the ventricular septum 408 (e.g., located at or around the RBB). In an embodiment, the ring electrode described herein can have a cylinder shape or be a closed coil or any other suitable shape. In an embodiment, the ring electrode 330 can have a length ranges from at or about 1 millimeter to at or about 4 millimeters (e.g., at or about 2 millimeters) to achieve high current density in pacing or the like.

Other embodiments of lead(s) can be found in the U.S. patent application Ser. No. 17/804,705, which hereby is incorporated herein by reference in its entirety.

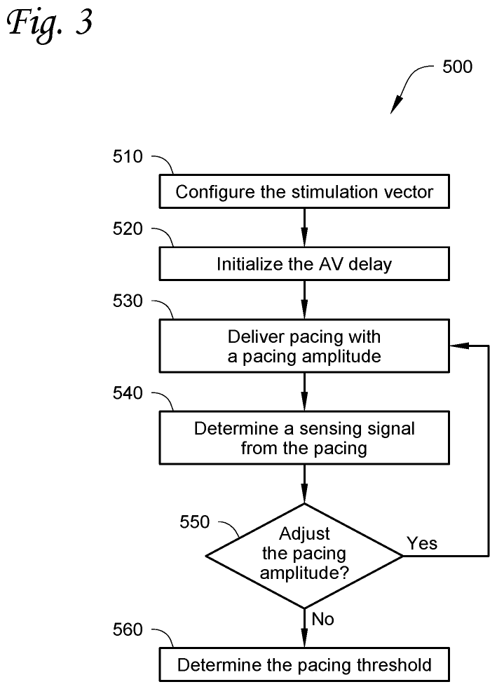

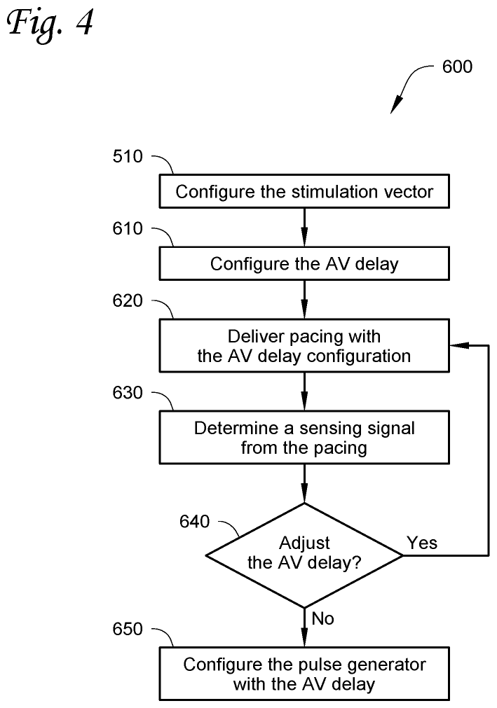

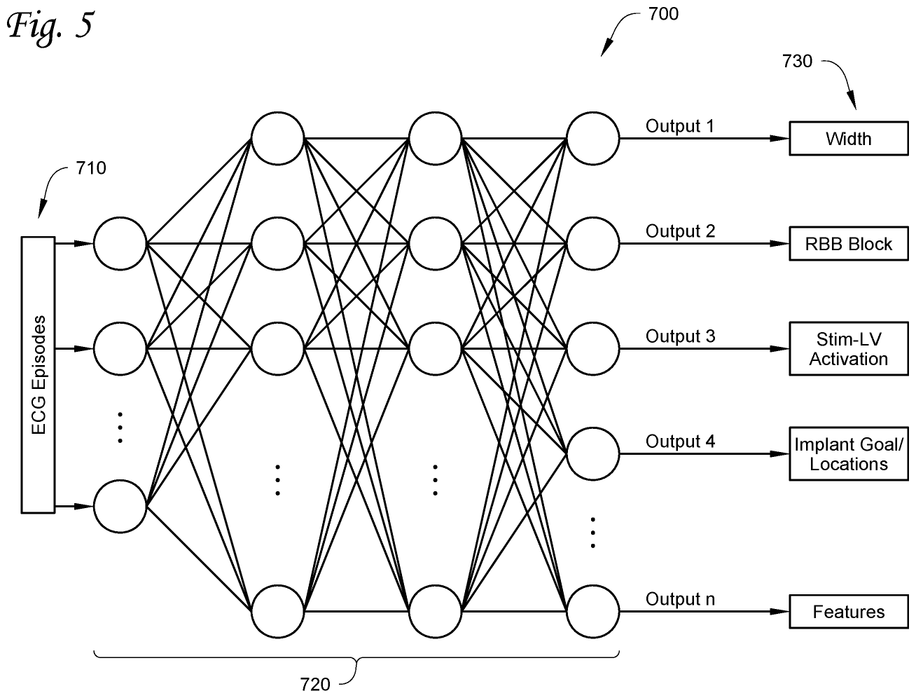

FIG. 3 is a flow chart illustrating a method 500 of determining a pacing threshold for the cardiac conduction system, according to an embodiment. FIG. 4 is a flow chart illustrating a method 600 of determining an AV delay for the cardiac conduction system, according to an embodiment. FIG. 5 illustrates a schematic view of a method 700 for a machine learning system for predicting and/or estimating parameters and/or configurations for the pulse generator and/or lead(s) for the cardiac conduction system, according to an embodiment.

It will be appreciated that the method steps disclosed herein can be conducted by a controller (e.g., the controller of a pulse generator such as an implantable pulse generator, the controller of a specially programmed computer e.g., used by a physician, or any suitable controller(s)), unless otherwise specified. The controller can include a processor, memory, and/or communication ports to communicate with e.g., other components of the pulse generator or specially programmed computer, and/or communicate with equipment or systems used before, during, and after implanting the pulse generator and/or the lead(s). The controller can communicate with other components using any suitable communications including wired and/or wireless, analog and/or digital communications. In an embodiment, the communication can include communications over telematics of the pulse generator or the specially programmed computer, which may be communicatively connected to telematics equipment, mobile device, communication system, cloud, or the like. The pulse generator or the specially programmed computer can include sensors (e.g., sound, acceleration, temperature, pressure, motion, voltage, current, battery status, battery charging level, or the like), or the pulse generator or the specially programmed computer can communicate with such sensors. The controller can obtain data sensed by the sensors and control the settings of the sensors and/or the components of the pulse generator or the specially programmed computer.

It will also be appreciated that the method(s) can include one or more operations, actions, or functions depicted by one or more blocks. Although illustrated as discrete blocks, various blocks may be divided into additional blocks, combined into fewer blocks, or eliminated, depending on the desired implementation.

As shown in FIG. 3, the method 500 begins at 510. At 510, the controller is configured to configure or set a stimulation vector (e.g., a pacing vector or the like) for a lead. The lead can be the lead 400 of FIG. 1 or FIG. 2 or any suitable lead. The lead includes a first electrode (e.g., 460 of FIG. 1 or FIG. 2) and a second electrode (e.g., 420 of FIG. 1 or FIG. 2). The first electrode can be positioned at or near left bundle branch of the cardiac conduction system. The second electrode can be positioned at or near right bundle branch of the cardiac conduction system. In an embodiment, the lead further includes a third electrode (e.g., 330 of FIG. 2). The third electrode can be positioned on a lead body outside of the septum.

The stimulation vector of the lead can be configured as a first unipolar configuration (e.g., set/configure the first electrode as cathode, and set/configure the can (i.e., the metal enclosure) of the pulse generator as ground), a second unipolar configuration (e.g., set/configure the second electrode as cathode, and set/configure the can as ground), a third unipolar configuration (e.g., set/configure the first electrode as cathode, set/configure the second electrode as cathode, and set/configure the can as ground), a first bipolar configuration (e.g., set/configure the first electrode as cathode, set/configure the second electrode as anode, and set/configure the can as ground), and a second bipolar configuration (e.g., set/configure the first electrode as anode, set/configure the second electrode as cathode, and set/configure the can as ground).

It will be appreciated that when there is an anode (e.g., in bipolar configurations), the stimulation vector can be from the cathode to the anode. When there is no anode (e.g., in unipolar configurations), the stimulation vector can be from the cathode to the ground/can.

In an embodiment, when the patient has LBBB, the controller can be configured to set/configure the stimulation vector as the first unipolar configuration (e.g., set/configure the first electrode as cathode, and set/configure the can as ground), set/configure the first electrode as a pacing electrode (with stimulation vector from the first electrode to the can) to deliver pacing to the LBB, and set/configure the second electrode as a sensing electrode (with sensing vector from the second electrode to the can) to conduct sensing of heart electrical signal(s).

In another embodiment, when the patient has LBBB, the controller can be configured to set/configure the stimulation vector as the first unipolar configuration (e.g., set/configure the first electrode as cathode, and set/configure the can as ground), set/configure the first electrode as a pacing electrode (with stimulation vector from the first electrode to the can) to deliver pacing to the LBB, and set/configure the third electrode as a sensing electrode (with sensing vector from the third electrode to the can) to conduct sensing of heart electrical signal(s).

In an embodiment, when the patient has RBBB, the controller can be configured to set/configure the stimulation vector as the second unipolar configuration (e.g., set/configure the second electrode as cathode, and set/configure the can as ground), set/configure the second electrode as a pacing electrode (with stimulation vector from the second electrode to the can) to deliver pacing to the RBB, and set/configure the first electrode as a sensing electrode (with sensing vector from the first electrode to the can) to conduct sensing of heart electrical signal(s).

In another embodiment, when the patient has RBBB, the controller can be configured to set/configure the stimulation vector as the second unipolar configuration (e.g., set/configure the second electrode as cathode, and set/configure the can as ground), set/configure the second electrode as a pacing electrode (with stimulation vector from the second electrode to the can) to deliver pacing to the RBB, and set/configure the third electrode as a sensing electrode (with sensing vector from the third electrode to the can) to conduct sensing of heart electrical signal(s).

It will be appreciated that the configured sensing electrode(s) described herein, which is not involved in pacing, can sense or detect sensing signals, and can provide better accuracy and be more reliable than sensing signals detected from the same pacing electrode that is reused as a sensing electrode. It will also be appreciated that the third electrode described herein can be used for activation determination and to safely and accurately determine the sensing signals.

At, before, or after 510, other method steps can be performed. The steps can include the controller initializing/setting/configuring an amplitude for the pacing (i.e., the pacing amplitude) by setting the pacing amplitude to a predetermined or desired value so that pacing stimulus can be delivered with such amplitude. In an embodiment, the pacing amplitude can be set as a maximum amplitude (e.g., at or about 3.5 volts or any suitable voltage) that ensure capture (or referred to as electrical capture). It will be appreciated that capture occurs when a pacing stimulus leads to depolarization of the heart (e.g., ventricle(s) or the like), which can be confirmed by electrocardiogram, e.g., a QRS complex and with a T wave, after each pacing stimulus. Loss of capture, also known as non-capture, is when the myocardium does not respond to the electrical pacing stimuli. In another embodiment, the pacing amplitude can be set as a minimum amplitude (e.g., at or about 0.2 volts or any suitable voltage) to begin with to e.g., minimize electrical energy used for pacing. The steps can also include determining an intrinsic AV delay. The intrinsic AV delay can be determined using any suitable means. For example, the intrinsic AV delay can be determined based on intrinsic (i.e., not stimulated by pacing) electrocardiogram sensed (e.g., the P wave to R wave interval, or the PR interval) or detected using sensing electrode(s) or other lead(s) (e.g., based on standard 12-lead electrocardiogram or the like).

The method 500 proceeds to 520. At 520, the controller is configured to initialize (or set or configure) an AV delay (e.g., a programmable AV delay for ventricular pacing or cardiac conduction system pacing). In an embodiment, the AV delay can be set as a delay that is less than the intrinsic AV delay for early capture on the pacing. In an embodiment, the AV delay to be at or about 80 percent of the determined intrinsic AV delay. In another embodiment, the AV delay to be at or about 60 percent or at or about 70 percent or any other suitable percentage of the determined intrinsic AV delay.

The method 500 proceeds to 530. At 530, the controller is configured to control the pulse generator to deliver a pacing with an configured pacing amplitude (e.g., the maximum pacing amplitude for stepping down or the minimum pacing amplitude for stepping up, for the first iteration of pacing), or with an adjusted pacing amplitude from 550, using the configured pacing electrode and stimulation vector.

The method 500 proceeds to 540. At 540, the controller is configured to determine a sensing signal from artifacts (e.g., stimulated atrial and/or ventricular depolarization signals or the like) of the pacing. The sensing signal can be sensed by e.g., the configured sensing electrode, and communicated to the controller.

The method 500 proceeds to 550. At 550, the controller is configured to determine whether or not to adjust the pacing amplitude based on the determined sensing signal at 540 and a capture signal. In an embodiment, each of the determined sensing signal and the capture signal can be electrocardiogram morphology (e.g., QRS morphology or the like). It will be appreciated that morphology can include the shape, duration, and/or amplitude shown in the electrocardiogram. In another embodiment, each of the determined sensing signal and the capture signal can be electrogram morphology (e.g., QRS morphology or the like). It will be appreciated that morphology can include the shape, duration, and/or amplitude shown in the electrogram. In another embodiment, each of the determined sensing signal and the capture signal can be intervals, duration, and/or amplitude (e.g., of the QRS or the like). In an embodiment, the capture signal can be determined using any suitable means. For example, the capture signal can be determined based on sensing the artifacts of a pacing that ensures capture.

At 550, the determined sensing signal matching the capture signal can be e.g., the intervals, duration, amplitude, and/or morphology of the determined sensing signal matching (e.g., equal to, exactly the same, or the like) the intervals, duration, amplitude, and/or morphology of the capture signal plus or minus a predetermined margin. The determined sensing signal not matching the capture signal can be e.g., the intervals, duration, amplitude, and/or morphology of the determined sensing signal not matching the intervals, duration, amplitude, and/or morphology of the capture signal plus or minus a predetermined margin.

In another embodiment, the correlation coefficient of the sensing signal with the stored or predetermined captured signal can be determined. The correlation coefficient of the sensing signal with the stored or predetermined non-captured signal can also be determined. The determined sensing signal matching the capture signal can be e.g., that the sensing signal has a higher correlation coefficient with the stored captured signal than the correlation coefficient of the sensing signal with the stored non-captured signal. The determined sensing signal not matching the capture signal can be e.g., that the sensing signal has a lower correlation coefficient with the stored captured signal than the correlation coefficient of the sensing signal with the stored non-captured signal. It will be appreciated that the correlation coefficient can be referred to as a statistical measure of the strength of the relationship between the relative movements of two variables.

At 550, adjusting the pacing amplitude can include increasing the pacing amplitude (e.g., when a minimum pacing amplitude is initialized/configured) by an amplitude (e.g., at or about 0.1 volts or the like) when the determined sensing signal does not match the capture signal (indicating a loss of capture), and the method 500 proceeds back to 530. When the determined sensing signal matches the capture signal, no increasing of the pacing amplitude may be performed (i.e., the adjustment is zero), the method 500 proceeds to 560 where the controller can be configured to set the pacing threshold as the configured pacing amplitude (for matching in the first iteration of pacing, where the adjustment is zero) or the increased/adjusted pacing amplitude.

At 550, adjusting the pacing amplitude can include decreasing the pacing amplitude (e.g., when a maximum pacing amplitude is initialized/configured) by an amplitude (e.g., at or about 0.1 volts or the like) when the determined sensing signal matches the capture signal, and the method 500 proceeds back to 530. When the determined sensing signal does not match the capture signal (indicating a loss of capture), no decreasing of the pacing amplitude may be further performed (i.e., the adjustment is zero), the method 500 proceeds to 560 where the controller can be configured to set the pacing threshold as the pacing amplitude right before being decreased/adjusted (to ensure capture) in the most recent iteration.

It will be appreciated that at 550, error handling (e.g., issuing an alert, stopping/exiting the method, or the like) can be applied when the adjusted pacing amplitude exceeds a maximum allowable amplitude or is below a minimum safety amplitude.

It will also be appreciated that the method 500 of FIG. 3 can similarly be used for determining and configuring other parameters such as sensing threshold, or the like.

As shown in FIG. 4, the method 600 begins at 510, which is the same as 510 of FIG. 3. The method 600 proceeds to 610.

At 610, the controller is configured to configure/set the AV delay (i.e., the programmable AV delay) to a fraction or percentage of the intrinsic AV delay or to the adjusted AV delay from 640. The intrinsic AV delay can be determined at, before, or after 510 as described in FIG. 3. Referring back to FIG. 4, in an embodiment, the AV delay can be set to at or about 100 percent of the determined intrinsic AV delay. In another embodiment, the AV delay can be set to at or about 90 percent, at or about 110 percent, or any other suitable percentage of the determined intrinsic AV delay. The method 600 proceeds to 620.

At 620, the controller is configured to control the pulse generator to deliver a pacing with the AV delay configured from 610, using the configured pacing electrode and stimulation vector from 510. The method 600 proceeds to 630.

At 630, the controller is configured to determine a sensing signal from artifacts of the pacing. The sensing signal can be sensed by e.g., the configured sensing electrode from 510, and communicated to the controller. The method 600 proceeds to 640.

At 640, the controller is configured to determine whether or not to adjust the AV delay based on whether or not the determined sensing signal at 630 has a minimal total ventricular activation time (TVAT) (by e.g., comparing the TVAT determined based on the determined sensing signal with a pre-stored or predetermined TVAT). It will be appreciated that a TVAT can be defined as a QRS duration (e.g., from the electrocardiogram). For example, a TVAT can be the time between the beginnings of the QRS deflection to the peak R, which can be in the range of at or about 35 milliseconds to at or about 40 milliseconds. It will be appreciated that in the first iteration, the initial TVAT can be determined by pacing the ventricle with an AV delay at or about 60% intrinsic AV delay configuration, which may result in a maximum TVAT (which can be determined based on the determined sensing signal for the pacing artifacts) that can be used as the initial TVAT. In an embodiment, the determined sensing signal can be electrocardiogram morphology (e.g., QRS morphology or the like) and/or electrogram morphology. It will be appreciated that morphology can include the shape, duration, and/or amplitude of the electrocardiogram. In another embodiment, the determined sensing signal can be intervals, duration, and/or amplitude (e.g., of the QRS or the like).

At 640, in the first iteration of the method, the pre-stored TVAT can be determined using any suitable means and stored in the memory (e.g., of the controller or the like). For example, the pre-stored TVAT can be determined based on intrinsic electrocardiogram sensed or detected using sensing electrode(s) or other lead(s) (e.g., based on standard 12-lead electrocardiogram or the like).

At 640, when the determined sensing signal from 630 has a minimal TVAT (e.g., the TVAT determined based on the determined sensing signal is not less than the pre-stored TVAT), no adjustment of the AV delay may be performed (i.e., the adjustment is zero), and the method 600 proceeds to 650 where the controller is configured to configure the pulse generator with the AV delay.

At 640, when the determined sensing signal from 630 does not have a minimal TVAT (e.g., the TVAT determined based on the determined sensing signal is less than the pre-stored TVAT) yet, the controller is configured to adjust the AV delay by a delay (e.g., at or about 10 milliseconds or any other suitable delay). In an embodiment, adjusting the AV delay includes increasing the AV delay by such delay. It will be appreciated that if the AV delay is decreased instead, the TVAT may increase which may result in less efficient contraction. The TVAT determined based on the determined sensing signal is now set as the new pre-stored TVAT, to replace the existing pre-stored TVAT. Then the method 600 proceeds back to 610 where the controller is configured to configure/set the AV delay to the adjusted AV delay from 640.

It will be appreciated that at 640, error handling (e.g., issuing an alert, stopping/exiting the method, or the like) can be applied when the adjusted AV delay exceeds a maximum allowable AV delay or is below a minimum allowable AV delay.

It will also be appreciated that the method 600 of FIG. 4 can similarly be used for determining and configuring other parameters such as interventricular delay, or the like.

It will further be appreciated that in the method 500 of FIG. 3 and in the method 600 of FIG. 4, there can be minimum required delay between pacing and sensing (e.g., sensing the artifacts of the pacing), and/or between pacing and next pacing.

In an embodiment, the method 500 of FIG. 3 and the method 600 of FIG. 4 can also include other method steps (e.g., conduced or performed by a controller of a specially programmed computer or the like). The steps can include sensing or detecting electrocardiogram. The electrocardiogram can be e.g., intrinsic (i.e., not stimulated by pacing) electrocardiogram. The electrocardiogram can be sensed or detected using sensing electrode(s) or other lead(s) (e.g., based on standard 12-lead electrocardiogram or the like). The steps can also include determining LBBB (or RBBB) based on the detected electrocardiogram and a LBBB (or RBBB) electrocardiogram (e.g., a pattern or electrocardiogram indicating LBBB (or RBBB)). In an embodiment, each of the detected electrocardiogram and the LBBB (or RBBB) electrocardiogram can be an electrocardiogram morphology.

The detected electrocardiogram matching (e.g., equal to, exactly the same, or the like) the LBBB (or RBBB) electrocardiogram can be e.g., the morphology of the detected electrocardiogram matching the morphology of the LBBB (or RBBB) electrocardiogram plus or minus a predetermined margin. The detected electrocardiogram not matching the LBBB (or RBBB) electrocardiogram can be e.g., the morphology of the detected electrocardiogram not matching the morphology of the LBBB (or RBBB) electrocardiogram plus or minus a predetermined margin. The detected electrocardiogram matching the LBBB electrocardiogram indicates that the patient has an LBBB, and/or the detected electrocardiogram matching the RBBB electrocardiogram indicates that the patient has a RBBB. Stimulation vector (e.g., at 510 of FIGS. 3 and 4) can be set based on whether the patient has an LBBB or a RBBB.

In another embodiment, the correlation coefficient of the detected electrocardiogram with the stored or predetermined LBBB (or RBBB) electrocardiogram can be determined. The correlation coefficient of the detected electrocardiogram with the stored or predetermined non-LBBB (or non-RBBB) electrocardiogram can also be determined. The detected electrocardiogram matching the LBBB (or RBBB) electrocardiogram can be e.g., that the detected electrocardiogram has a higher correlation coefficient with the stored LBBB (or RBBB) electrocardiogram than the correlation coefficient of the detected electrocardiogram with the stored non-LBBB (or non-RBBB) electrocardiogram. The detected electrocardiogram not matching the LBBB (or RBBB) electrocardiogram can be e.g., that the detected electrocardiogram has a lower correlation coefficient with the stored LBBB (or RBBB) electrocardiogram than the correlation coefficient of the detected electrocardiogram with the stored non-LBBB (or non-RBBB) electrocardiogram. It will be appreciated that the correlation coefficient can be referred to as a statistical measure of the strength of the relationship between the relative movements of two variables.

FIG. 5 illustrates a schematic view of a method 700 for a machine learning system for predicting and/or estimating parameters and/or configurations for the pulse generator and/or lead(s) for the cardiac conduction system, according to an embodiment.

The method 700 may include one or more operations, actions, or functions 720. The method 700 can deploy e.g., a trained machine learning model (e.g., an electrocardiogram model, a sensor model, or the like) to determine parameters and/or configurations for the pulse generator and/or lead(s) for the cardiac conduction system. For example, the method can include determining or predicting the pulse generator and/or lead(s) configurations based on inputs such as electrocardiogram using machine learning technologies.

As shown in FIG. 5, the method 700 includes collecting input data 710 such as the sensed or detected electrocardiogram data (e.g., QRS morphologies or the like) and/or sensed data (e.g., heart sound, impedance, or the like, from sensors) to the electrocardiogram model (or sensor model). The running of the trained electrocardiogram model (or trained sensor model) (e.g., by the controller) can provide outputs 730 such as the QRS duration/width, RBBB morphologies, TVAT, stimulated LV activation, desired lead electrode implant locations (e.g., with real time interpretation), impedance such as beat-to-beat impedance, or the like.

It will be appreciated that the method 700 can include steps 720 such as the controller creating the machine learning model (e.g., the electrocardiogram model, the sensor model, or the like). The machine learning model can be saved in e.g., a memory or any other suitable devices. It will be appreciated that the method 700 can also include steps 720 such as the controller training the machine learning model using data from 710. It will be appreciated that the method 700 can further include steps 720 such as the controller deploying the trained machine learning model for use. For example, the trained machine learning model can be deployed to a controller in the field for use. It will be appreciated that the type and/or source of the data for running the trained machine learning model can be similar to the type and/or source of the data for training the machine learning model. It will be appreciated that the method 700 can also include steps 720 such as the controller re-training the machine learning model using updated or new training data. It will also be appreciated that neural network such as convolutional neural network or any other suitable machine learning tool(s) can be used as a tool for the machine learning process. It will be appreciated that the method 700 may be in addition to or may replace some blocks of FIGS. 3 and/or 4 to serve as an alternative embodiment. For example, in FIGS. 3 and/or 4, when the electrocardiogram is sensed or detected, the electrocardiogram data (for a particular patient and/or for a group of patients) can be saved or collected into a data repository (e.g., a database or the like) at e.g., 710. Then the method steps 720 can be performed, and at 730 the AV delay for 650 of FIG. 4, the pacing threshold for 560 of FIG. 3, or the like, can be predicted as output(s) of the method 700.

It will also be appreciated that preferably, personalized machine learning model (e.g., electrocardiogram or sensor data collected for a particular patient) may be used to predict parameters and/or configurations for that particular patient to provide more accuracy. Preferably, intra-cardiac electrocardiogram (IEGM) data and/or other sensor data may be collected/used at 710. Preferably, Bluetooth low energy communication protocol may be used for communicating data or communicating control settings (e.g., parameters, configurations, etc.).

Aspects:

It is appreciated that any one of aspects can be combined with other aspect(s).
Aspect 1. An implantable pulse generator for cardiac conduction system, the pulse generator comprising:
an enclosure containing an electronic circuitry having a controller;
wherein the controller is configured to:
   configure a stimulation vector for a lead, the lead including a first electrode and a second electrode, the first electrode being positioned at or near left bundle branch of the cardiac conduction system, the second electrode being positioned at or near right bundle branch of the cardiac conduction system,
   configure an atrioventricular (AV) delay to a first delay, the first delay being less than an intrinsic AV delay,
   control the pulse generator to deliver pacing with a pacing amplitude,
   determine a sensing signal from artifacts of the pacing,
   adjust the pacing amplitude by a first amplitude based on the determined sensing signal and a capture signal, and
   determine a pacing threshold based on the adjusted pacing amplitude.
Aspect 2. The pulse generator according to aspect 1, wherein the first delay is at or about 80 percent of the intrinsic AV delay.
Aspect 3. The pulse generator according to aspect 1 or aspect 2, wherein the first amplitude is at or about 0.1 volts.
Aspect 4. The pulse generator according to any one of aspects 1-3, wherein the controller adjusting the pacing amplitude includes increasing the pacing amplitude by the first amplitude when the determined sensing signal does not match the capture signal,
when the determined sensing signal matches the capture signal, the pacing threshold is set as the adjusted pacing amplitude.
Aspect 5. The pulse generator according to any one of aspects 1-4, wherein the controller adjusting the pacing amplitude includes decreasing the pacing amplitude by the first amplitude when the determined sensing signal matches the capture signal,
when the determined sensing signal does not match the capture signal, the pacing threshold is set as the pacing amplitude before being adjusted.
Aspect 6. The pulse generator according to any one of aspects 1-5, wherein the controller is further configured to set the first electrode as a pacing electrode and set the second electrode as a sensing electrode for left bundle branch block.
Aspect 7. The pulse generator according to any one of aspects 1-6, wherein the controller is further configured to set the second electrode as a pacing electrode and set the first electrode as a sensing electrode for right bundle branch block.
Aspect 8. The pulse generator according to any one of aspects 1-7, wherein the first electrode is a linear electrode, and the second electrode is a helix electrode.
Aspect 9. The pulse generator according to any one of aspects 1-8, wherein the determined sensing signal includes a first QRS morphology and the capture signal includes a second QRS morphology.
Aspect 10. The pulse generator according to any one of aspects 1-9, wherein the determined sensing signal includes a first QRS interval and the capture signal includes a second QRS interval.
Aspect 11. The pulse generator according to any one of aspects 1-10, wherein the lead further includes a third electrode, the third electrode being positioned on a lead body outside of a septum.
Aspect 12. The pulse generator according to aspect 11, wherein the controller is further configured to set the first electrode as a pacing electrode and set the third electrode as a sensing electrode for left bundle branch block.
Aspect 13. The pulse generator according to aspect 11, wherein the controller is further configured to set the second electrode as a pacing electrode and set the third electrode as a sensing electrode for right bundle branch block.
Aspect 14. The pulse generator according to any one of aspects 11-13, wherein the first electrode is a linear electrode, the second electrode is a helix electrode, and the third electrode is a ring electrode.
Aspect 15. A method of determining a pacing threshold for cardiac conduction system, the method comprising:
configuring a stimulation vector for a lead, the lead including a first electrode and a second electrode, the first electrode being positioned at or near left bundle branch of the cardiac conduction system, the second electrode being positioned at or near right bundle branch of the cardiac conduction system,
configuring an atrioventricular (AV) delay to a first delay, the first delay being less than an intrinsic AV delay,
controlling a pulse generator to deliver pacing with a pacing amplitude,
determining a sensing signal from artifacts of the pacing,
adjusting the pacing amplitude by a first amplitude based on the determined sensing signal and a capture signal, and
determining the pacing threshold based on the adjusted pacing amplitude.
Aspect 16. The method according to aspect 15, wherein configuring the AV delay to the first delay includes configuring the AV delay to be at or about 80 percent of the intrinsic AV delay.
Aspect 17. The method according to aspect 15 or aspect 16, wherein adjusting the pacing amplitude by the first amplitude includes adjusting the pacing amplitude by at or about 0.1 volts.
Aspect 18. The method according to any one of aspects 15-17, wherein adjusting the pacing amplitude includes increasing the pacing amplitude by the first amplitude when the determined sensing signal does not match the capture signal,
the method further comprising:
when the determined sensing signal matches the capture signal, setting the pacing threshold as the adjusted pacing amplitude.
Aspect 19. The method according to any one of aspects 15-18, wherein adjusting the pacing amplitude includes decreasing the pacing amplitude by the first amplitude when the determined sensing signal matches the capture signal,
the method further comprising:
when the determined sensing signal does not match the capture signal, setting the pacing threshold as the pacing amplitude before being adjusted.
Aspect 20. The method according to any one of aspects 15-19, further comprising: setting the first electrode as a pacing electrode and setting the second electrode as a sensing electrode for left bundle branch block.
Aspect 21. The method according to any one of aspects 15-20, further comprising:
setting the second electrode as a pacing electrode and setting the first electrode as a sensing electrode for right bundle branch block.
Aspect 22. The method according to any one of aspects 15-21, wherein the first electrode is a linear electrode, and the second electrode is a helix electrode.
Aspect 23. The method according to any one of aspects 15-22, wherein the determined sensing signal includes a first QRS morphology and the capture signal includes a second QRS morphology.
Aspect 24. The method according to any one of aspects 15-23, wherein the determined sensing signal includes a first QRS interval and the capture signal includes a second QRS interval.
Aspect 25. The method according to any one of aspects 15-24, wherein the lead further includes a third electrode, the third electrode being positioned on a lead body outside of a septum.
Aspect 26. The method according to aspect 25, further comprising:
setting the first electrode as a pacing electrode and setting the third electrode as a sensing electrode for left bundle branch block.
Aspect 27. The method according to aspect 25, further comprising:
setting the second electrode as a pacing electrode and setting the third electrode as a sensing electrode for right bundle branch block.
Aspect 28. The method according to any one of aspects 25-27, wherein the first electrode is a linear electrode, the second electrode is a helix electrode, and the third electrode is a ring electrode.
Aspect 29. The method according to any one of aspects 15-28, further comprising:
detecting an electrocardiogram; and
determining left bundle branch block based on the electrocardiogram and a left bundle branch block electrocardiogram.
Aspect 30. The method according to any one of aspects 15-29, further comprising:
detecting an electrocardiogram; and
determining right bundle branch block based on the electrocardiogram and a right bundle branch block electrocardiogram.
Aspect 31. The method according to any one of aspects 15-30, further comprising:
detecting an electrocardiogram; and
determining pulse generator configurations based on the electrocardiogram using machine learning.
Aspect 32. An implantable pulse generator for cardiac conduction system, the pulse generator comprising:
an enclosure containing an electronic circuitry having a controller;
wherein the controller is configured to:
   configure a stimulation vector for a lead, the lead including a first electrode and a second electrode, the first electrode being positioned at or near left bundle branch of the cardiac conduction system, the second electrode being positioned at or near right bundle branch of the cardiac conduction system,
   configure an atrioventricular (AV) delay to a percentage of an intrinsic AV delay,
   control the pulse generator to deliver pacing with the AV delay,
   determine a sensing signal from artifacts of the pacing,
   adjust the AV delay by a first delay based on the determined sensing signal, and
   configure the pulse generator with the adjusted AV delay.
Aspect 33. The pulse generator according to aspect 32, wherein the first delay is at or about 10 milliseconds.
Aspect 34. The pulse generator according to aspect 32 or aspect 33, wherein the percentage is 100 percent.
Aspect 35. The pulse generator according to any one of aspects 32-34, wherein the controller adjusting the AV delay includes increasing the AV delay by the first delay when a total ventricular activation time of the determined sensing signal is less than a pre-stored duration,
when the total ventricular activation time of the determined sensing signal is not less than the pre-stored duration, the controller is configured to configure the pulse generator with the adjusted AV delay.
Aspect 36. The pulse generator according to any one of aspects 32-35, wherein the controller is further configured to set the first electrode as a pacing electrode and set the second electrode as a sensing electrode for left bundle branch block.
Aspect 37. The pulse generator according to any one of aspects 32-36, wherein the controller is further configured to set the second electrode as a pacing electrode and set the first electrode as a sensing electrode for right bundle branch block.
Aspect 38. The pulse generator according to any one of aspects 32-37, wherein the first electrode is a linear electrode, and the second electrode is a helix electrode.
Aspect 39. The pulse generator according to any one of aspects 32-38, wherein the determined sensing signal includes a QRS morphology.
Aspect 40. The pulse generator according to any one of aspects 32-39, wherein the determined sensing signal includes a QRS interval.
Aspect 41. The pulse generator according to any one of aspects 32-40, wherein the lead further includes a third electrode, the third electrode being positioned on a lead body outside of a septum.
Aspect 42. The pulse generator according to aspect 41, wherein the controller is further configured to set the first electrode as a pacing electrode and set the third electrode as a sensing electrode for left bundle branch block.
Aspect 43. The pulse generator according to aspect 41, wherein the controller is further configured to set the second electrode as a pacing electrode and set the third electrode as a sensing electrode for right bundle branch block.
Aspect 44. The pulse generator according to any one of aspects 41-43, wherein the first electrode is a linear electrode, the second electrode is a helix electrode, and the third electrode is a ring electrode.
Aspect 45. A method of determining an atrioventricular (AV) delay for cardiac conduction system, the method comprising:
configuring a stimulation vector for a lead, the lead including a first electrode and a second electrode, the first electrode being positioned at or near left bundle branch of the cardiac conduction system, the second electrode being positioned at or near right bundle branch of the cardiac conduction system,
configuring the AV delay to a percentage of an intrinsic AV delay,
controlling the pulse generator to deliver pacing with the AV delay,
determining a sensing signal from artifacts of the pacing,
adjusting the AV delay by a first delay based on the determined sensing signal, and
configuring the pulse generator with the adjusted AV delay.
Aspect 46. The pulse generator according to aspect 45, wherein adjusting the AV delay by the first delay includes adjusting the AV delay by at or about 10 milliseconds.
Aspect 47. The method according to aspect 45 or aspect 46, wherein configuring the AV delay to the percentage of the intrinsic AV delay includes configuring the AV delay to 100 percent of the intrinsic AV delay.
Aspect 48. The method according to any one of aspects 45-47, wherein adjusting the AV delay includes increasing the AV delay by the first delay when a total ventricular activation time of the determined sensing signal is less than a pre-stored duration,
the method further comprising:
when the total ventricular activation time of the determined sensing signal is not less than the pre-stored duration, configuring the pulse generator with the adjusted AV delay.
Aspect 49. The method according to any one of aspects 45-48, further comprising:
setting the first electrode as a pacing electrode and setting the second electrode as a sensing electrode for left bundle branch block.
Aspect 50. The method according to any one of aspects 45-49, further comprising:
setting the second electrode as a pacing electrode and setting the first electrode as a sensing electrode for right bundle branch block.
Aspect 51. The method according to any one of aspects 45-50, wherein the first electrode is a linear electrode, and the second electrode is a helix electrode.
Aspect 52. The method according to any one of aspects 45-51, wherein the determined sensing signal includes a QRS morphology.
Aspect 53. The method according to any one of aspects 45-52, wherein the determined sensing signal includes a QRS interval.
Aspect 54. The method according to any one of aspects 45-53, wherein the lead further includes a third electrode, the third electrode being positioned on a lead body outside of a septum.
Aspect 55. The method according to aspect 54, further comprising:
setting the first electrode as a pacing electrode and setting the third electrode as a sensing electrode for left bundle branch block.
Aspect 56. The method according to aspect 54, further comprising:
setting the second electrode as a pacing electrode and setting the third electrode as a sensing electrode for right bundle branch block.
Aspect 57. The method according to any one of aspects 54-56, wherein the first electrode is a linear electrode, the second electrode is a helix electrode, and the third electrode is a ring electrode.
Aspect 58. The method according to any one of aspects 45-57, further comprising:
detecting an electrocardiogram; and
determining left bundle branch block based on the electrocardiogram and a left bundle branch block electrocardiogram.
Aspect 59. The method according to any one of aspects 45-58, further comprising:
detecting an electrocardiogram; and
determining right bundle branch block based on the electrocardiogram and a right bundle branch block electrocardiogram.
Aspect 60. The method according to any one of aspects 45-59, further comprising:
detecting an electrocardiogram; and
determining pulse generator configurations based on the electrocardiogram using machine learning.

The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

What is claimed is:

1. An implantable pulse generator for cardiac conduction system, the pulse generator comprising:
an enclosure containing an electronic circuitry having a controller;
the controller is configured to:
configure a stimulation vector for a lead, the lead including a first electrode and a second electrode, the first electrode being positioned at or near left bundle branch of the cardiac conduction system, the second electrode being positioned at or near right bundle branch of the cardiac conduction system,
configure an atrioventricular (AV) delay to a first delay, the first delay being less than an intrinsic AV delay,
control the pulse generator to deliver pacing with a pacing amplitude,
determine a sensing signal from artifacts of the pacing,
adjust the pacing amplitude by a first amplitude based on the determined sensing signal and a capture signal, and
determine a pacing threshold based on the adjusted pacing amplitude.

2. The pulse generator according to claim 1, wherein the first delay is at or about 80 percent of the intrinsic AV delay.

3. The pulse generator according to claim 1, wherein the first amplitude is at or about 0.1 volts.

4. The pulse generator according to claim 1, wherein the controller adjusting the pacing amplitude includes increasing the pacing amplitude by the first amplitude when the determined sensing signal does not match the capture signal,
when the determined sensing signal matches the capture signal, the pacing threshold is set as the adjusted pacing amplitude.

5. The pulse generator according to claim 1, wherein the controller adjusting the pacing amplitude includes decreasing the pacing amplitude by the first amplitude when the determined sensing signal matches the capture signal,
when the determined sensing signal does not match the capture signal, the pacing threshold is set as the pacing amplitude before being adjusted.

6. The pulse generator according to claim 1, wherein the lead further includes a third electrode being positioned on a lead body.

7. The pulse generator according to claim 6, wherein the first electrode is a linear electrode, the second electrode is a helix electrode, and the third electrode is a ring electrode.

8. A method of determining a pacing threshold for cardiac conduction system, the method comprising:
configuring a stimulation vector for a lead, the lead including a first electrode and a second electrode, the first electrode being positioned at or near left bundle branch of the cardiac conduction system, the second electrode being positioned at or near right bundle branch of the cardiac conduction system,
configuring an atrioventricular (AV) delay to a first delay, the first delay being less than an intrinsic AV delay,
controlling a pulse generator to deliver pacing with a pacing amplitude,
determining a sensing signal from artifacts of the pacing,
adjusting the pacing amplitude by a first amplitude based on the determined sensing signal and a capture signal, and
determining the pacing threshold based on the adjusted pacing amplitude.

9. The method according to claim 8, wherein configuring the AV delay to the first delay includes configuring the AV delay to be at or about 80 percent of the intrinsic AV delay.

10. The method according to claim 8, wherein adjusting the pacing amplitude by the first amplitude includes adjusting the pacing amplitude by at or about 0.1 volts.

11. The method according to claim 8, wherein adjusting the pacing amplitude includes increasing the pacing amplitude by the first amplitude when the determined sensing signal does not match the capture signal,
the method further comprising:
when the determined sensing signal matches the capture signal, setting the pacing threshold as the adjusted pacing amplitude.

12. The method according to claim 8, wherein adjusting the pacing amplitude includes decreasing the pacing amplitude by the first amplitude when the determined sensing signal matches the capture signal,
the method further comprising:
when the determined sensing signal does not match the capture signal, setting the pacing threshold as the pacing amplitude before being adjusted.

13. The method according to claim 8, wherein the lead further includes a third electrode positioned on a lead body.

14. The method according to claim 8, further comprising:
detecting an electrocardiogram or an electrogram; and
determining pulse generator configurations based on the electrocardiogram or the electrogram using machine learning.

15. A method of determining an atrioventricular (AV) delay for cardiac conduction system, the method comprising:
configuring a stimulation vector for a lead, the lead including a first electrode and a second electrode, the first electrode being positioned at or near left bundle branch of the cardiac conduction system, the second electrode being positioned at or near right bundle branch of the cardiac conduction system,
configuring the AV delay to a percentage of an intrinsic AV delay,
controlling the pulse generator to deliver pacing with the AV delay,
determining a sensing signal from artifacts of the pacing,
adjusting the AV delay by a first delay based on the determined sensing signal, and
configuring the pulse generator with the adjusted AV delay.

16. The pulse generator according to claim 15, wherein adjusting the AV delay by the first delay includes adjusting the AV delay by at or about 10 milliseconds.

17. The method according to claim 15, wherein configuring the AV delay to the percentage of the intrinsic AV delay includes configuring the AV delay to 100 percent of the intrinsic AV delay.

18. The method according to claim 15, wherein adjusting the AV delay includes increasing the AV delay by the first delay when a total ventricular activation time of the determined sensing signal is less than a pre-stored duration,
the method further comprising:
when the total ventricular activation time of the determined sensing signal is not less than the pre-stored duration, configuring the pulse generator with the adjusted AV delay.

19. The method according to claim 15, wherein the lead further includes a third electrode positioned on a lead body.

20. The method according to claim 15, further comprising:
detecting an electrocardiogram or an electrogram; and
determining pulse generator configurations based on the electrocardiogram or the electrogram using machine learning.
